(19) United States
(12) Patent Application Publication
(10) Pub. No.: US 2025/0358561 A1
(43) Pub. Date: Nov. 20, 2025

(54) ERGONOMIC EARPIECE AND METHODS

(71) Applicant: SureFire, LLC, Fountain Valley, CA (US)

(72) Inventors: Richard C. Smith, Costa Mesa, CA (US); Alfredo Soria, Corona, CA (US)

(21) Appl. No.: 19/278,612

(22) Filed: Jul. 23, 2025

Related U.S. Application Data
(63) Continuation of application No. PCT/US2025/019635, filed on Mar. 12, 2025.
(60) Provisional application No. 63/566,044, filed on Mar. 15, 2024.

(51) Int. Cl. H04R 1/10

(52) U.S. Cl. CPC H04R 1/105; H04R 1/1016

(57) ABSTRACT

An ergonomic earpiece for a dynamic fit in a wearer's ear and corresponding methods are disclosed. The earpiece may include an arcuate rib having upper and lower ends. The arcuate rib may be received by an antihelix and an antitragus of a wearer's ear. The earpiece may also include an upper lobe extending from the upper end of the arcuate rib, where the upper lobe is configured to be received in a cavity defined by a cymba concha and a crus of a helix of the ear. The earpiece may also include an aperture in the upper lobe configured to permit the upper lobe to exhibit a flexion in response to a compression of the upper lobe against the ear to dynamically fit the earpiece to the ear. Additional devices and methods are also provided.

### CROSS-REFERENCE TO RELATED APPLICATIONS

This application is a continuation of International Patent Application No. PCT/US2025/019635 filed Mar. 12, 2025 and entitled “ERGONOMIC EARPIECE AND METHODS”, which claims priority to and the benefit of U.S. Provisional Patent Application No. 63/566,044 filed Mar. 15, 2024 and entitled “ERGONOMIC EARPIECE AND METHODS”, all of which are hereby incorporated by reference in their entirety.

### TECHNICAL FIELD

The present invention relates generally to earpieces and, more specifically, to ergonomic earpieces and related methods.

### BACKGROUND

Earpieces are often used to facilitate hearing protection and/or two-way communication by users. For example, police officers, firefighters, military personnel, and ordinary citizens may use earpieces in conjunction with other communication equipment, filters, and/or other apparatus when performing various duties and operations.

Although many contemporary earpieces have proven generally suitable for their intended purposes, some may possess inherent deficiencies that detract from their overall effectiveness and desirability. For example, the shape of some earpieces may not be universally suitable for all ear types and result in discomfort of the user. As such, it is desirable to provide an earpiece that provides improved ergonomics to reduce discomfort while maintaining a secure fit in a user's ear.

### SUMMARY

Methods and systems are described herein for providing an ergonomic earpiece providing a diverse comfort fit. For, instance, earpiece may fit a wide variety of ear types due to a deformability of earpiece. In some embodiments, the earpiece may include an arcuate rib that has a curvature. The arcuate rib may be received by an antihelix and an antitragus of an ear. The earpiece may include an upper lobe having an aperture that extends from the arcuate rib. The upper lobe may be configured to flex when the upper lobe contacts (e.g., is received in) a cavity defined by a cymba concha and a crus of a helix of the ear. The flexion of the upper lobe, which is facilitated by the aperture, allows for the upper lobe to deform into a unique shape complementary to the user's ear. In other embodiments, the earpiece may also include an extension that may be received by the ear canal, which in tandem with the upper lobe, provides a dynamic clamp force that further secures the earpiece to the ear.

For instance, an earpiece may include an upper portion having an aperture that allows upper portion to flex in response to being inserted into a user's ear. In another instance, the earpiece may include a dynamic clamp force used to comfortably secure the earpiece in a user's ear.

In one embodiment, an earpiece is provided. The earpiece includes an arcuate rib having upper and lower ends, wherein the arcuate rib is configured to be received by an antihelix and an antitragus of an ear. The earpiece includes an upper lobe extending from the upper end of the arcuate rib and configured to be received in a cavity defined by a cymba concha and a crus of a helix of the ear. The earpiece includes an aperture in the upper lobe configured to permit the upper lobe to exhibit a flexion in response to a compression of the upper lobe against the ear to dynamically fit the earpiece to the ear.

In another embodiment, a method of securing an earpiece to an ear is provided. The earpiece includes an arcuate rib having upper and lower ends, an upper lobe extending from the upper end of the arcuate rib, and an aperture in the upper lobe. The method of securing the earpiece includes inserting the upper lobe into a cavity defined by a cymba concha and a crus of a helix of the ear, compressing the upper lobe against the ear to exhibit a flexion to dynamically fit the earpiece to the ear, and positioning the arcuate rib against an antihelix and an antitragus of the ear.

The scope of the present disclosure is defined by the claims, which are incorporated into this section by reference. A more complete understanding of embodiments of the present disclosure will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 illustrates a left rear perspective view of an earpiece in accordance with an embodiment of the present disclosure.

FIG. 2 illustrates a front perspective view of an earpiece in accordance with an embodiment of the present disclosure.

FIG. 3 illustrates a right front side view of an earpiece in accordance with an embodiment of the present disclosure.

FIG. 4 illustrates a rear perspective view of an earpiece in accordance with an embodiment of the present disclosure.

FIG. 5 illustrates a left side view of an earpiece in accordance with an embodiment of the present disclosure.

FIG. 6 illustrates a right side view of an earpiece in accordance with an embodiment of the present disclosure.

FIG. 7 illustrates a front perspective view of an earpiece in accordance with an embodiment of the present disclosure.

FIG. 8 illustrates a rear perspective view of an earpiece in accordance with an embodiment of the present disclosure.

FIG. 9 illustrates a top view perspective view of an earpiece in accordance with an embodiment of the present disclosure.

FIG. 10 illustrates a bottom view perspective view of an earpiece in accordance with an embodiment of the present disclosure.

FIG. 11 illustrates an earpiece in a rest state and also in a compressed state exhibiting one or more flexions accordance with an embodiment of the present disclosure.

FIG. 12A illustrates grab points on an ear in accordance with an embodiment of the present disclosure.

FIG. 12B illustrates an earpiece disposed at least partially within an ear of a user in accordance with an embodiment of the present disclosure.

FIG. 13 illustrates a is a left rear perspective view of the earpiece with an example extension passing through an aperture of the earpiece in accordance with an embodiment of the present disclosure.

FIG. 14 illustrates a front perspective view of the earpiece with the example extension in accordance with an embodiment of the present disclosure.

FIG. 15 illustrates a right front side view of the earpiece with the example extension in accordance with an embodiment of the present disclosure.

FIG. 16 illustrates a rear perspective view of the earpiece with the example extension in accordance with an embodiment of the present disclosure.

FIG. 17 illustrates a cross-sectional view of the earpiece of FIG. 4, as seen along the lines of the section 17-17 taken therein, in accordance with an embodiment of the present disclosure.

FIG. 18 illustrates a flow chart of a method of securing an earpiece to an ear of a user in accordance with an embodiment of the present disclosure.

Embodiments of the present disclosure and their advantages are best understood by referring to the detailed description that follows. It is noted that sizes of various components and distances between these components are not drawn to scale in the figures. It should be appreciated that like reference numerals are used to identify like elements illustrated in one or more of the figures.

### DETAILED DESCRIPTION

Methods and systems are described herein for providing ergonomic earpieces. For instance, an earpiece may include an upper portion having an aperture that allows upper portion to flex in response to being inserted into a user's ear. In another instance, the earpiece may include a dynamic clamp force used to comfortably secure the earpiece in a user's ear.

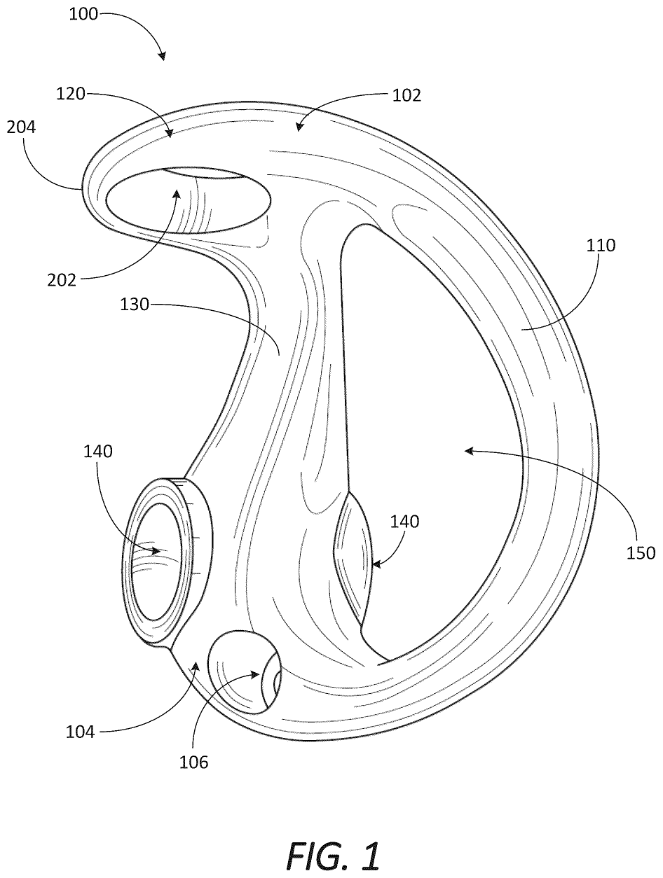

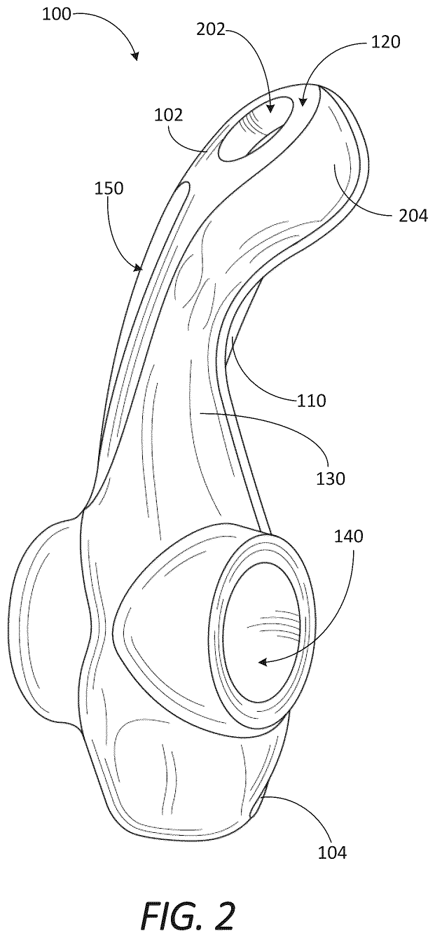

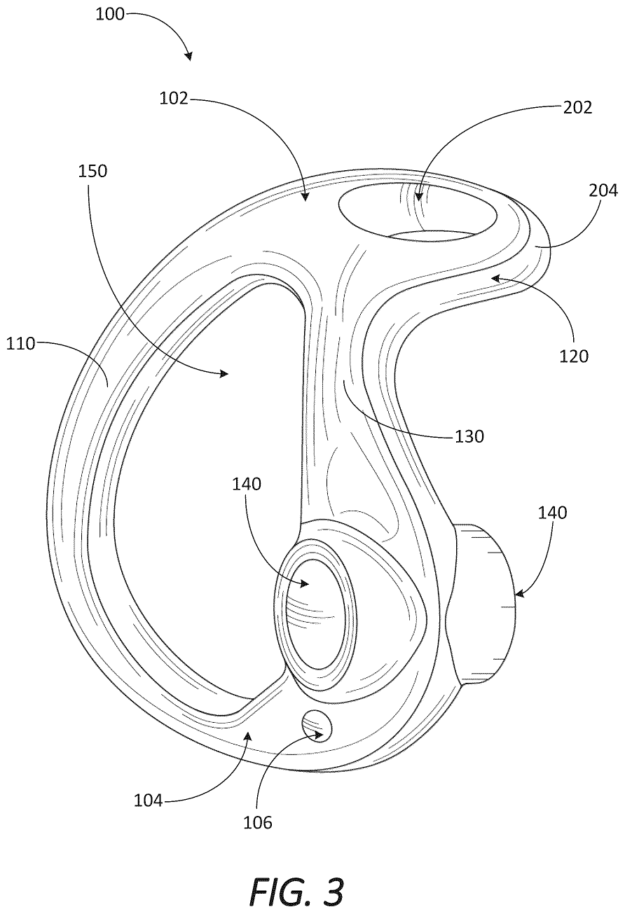

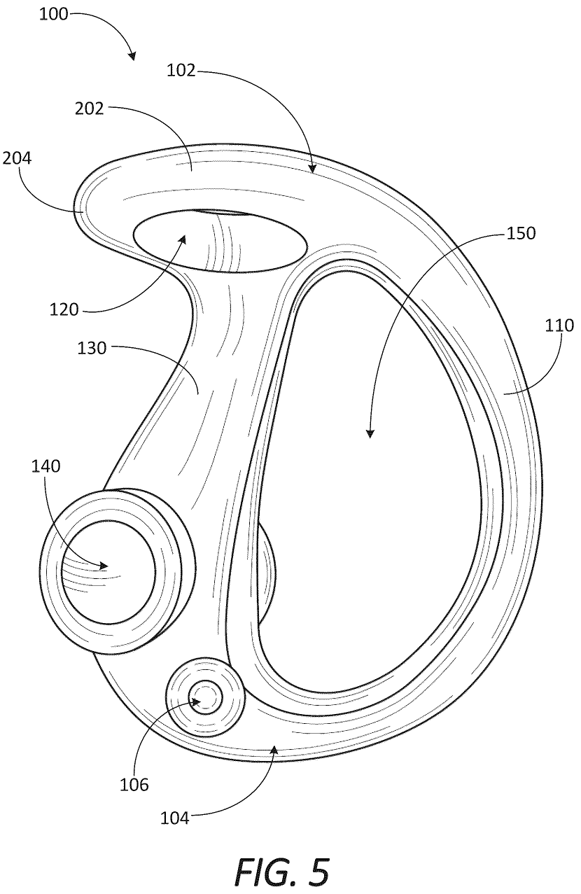

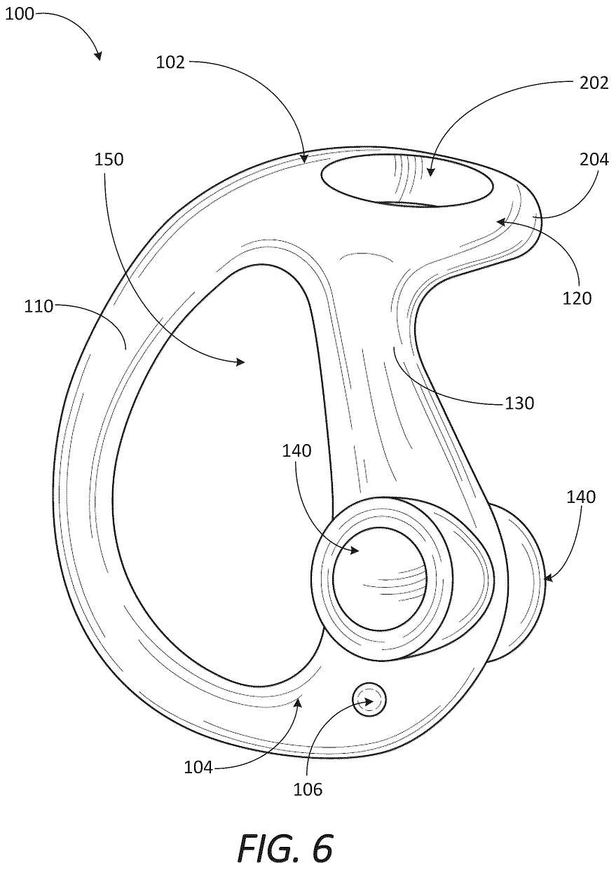

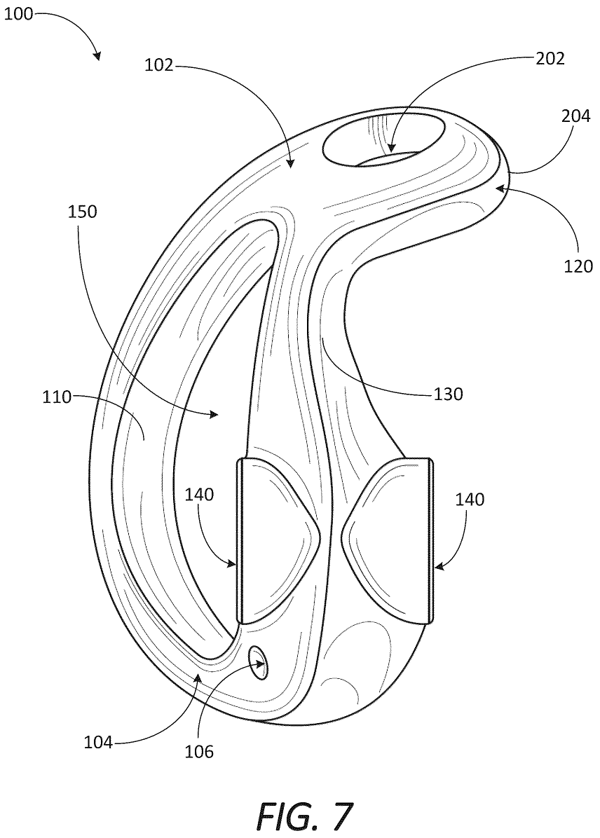

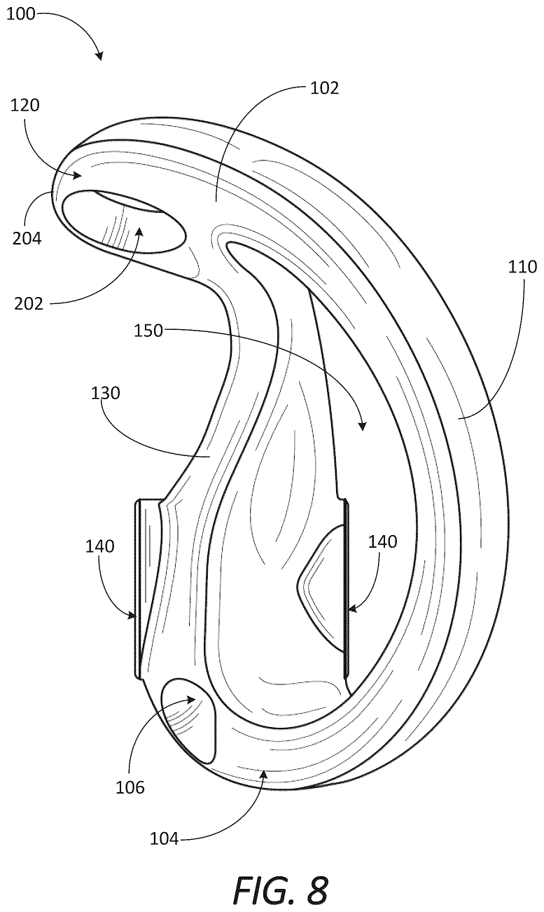

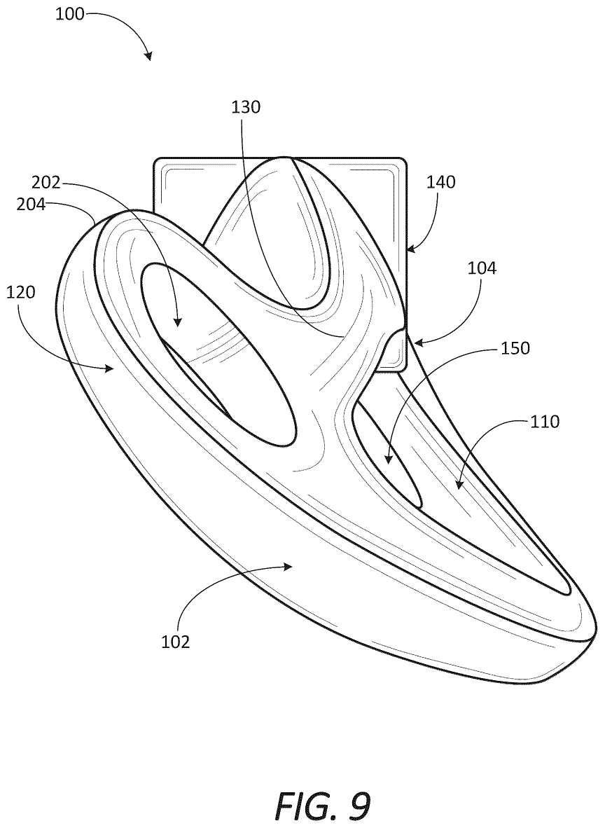

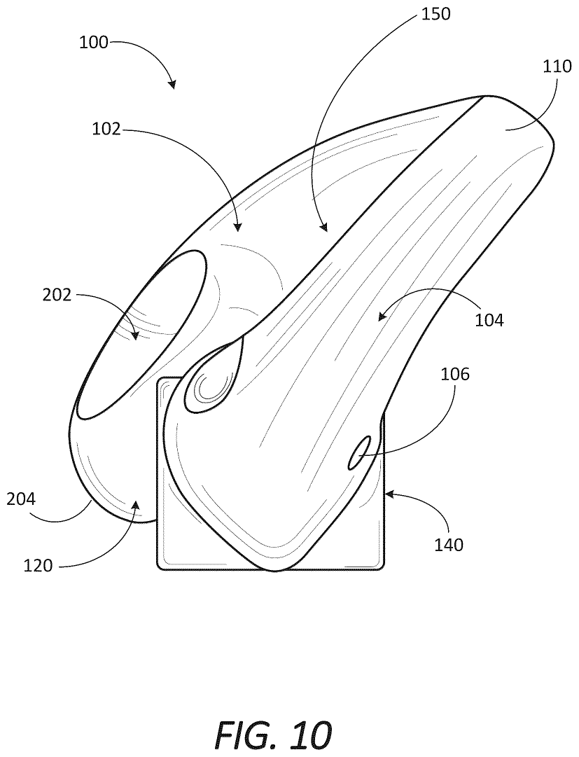

Referring now to FIGS. 1-10, various views of an ergonomic earpiece 100 is shown in accordance with embodiments of the present disclosure. In some embodiments, earpiece 100 is configured to fit within (e.g., be received by) a concha bowl (also referred to herein as a “concha”) of a user's ear. In various embodiments, ergonomic earpiece 100 (also referred to herein as an “earpiece”) includes an arcuate rib 110 having an upper end 102 and a lower end 104. Arcuate rib 110 may be configured to be received by an antihelix and an antitragus of a user's ear. In various embodiments, earpiece 100 may include an upper lobe 120 extending from upper end 102 of arcuate rib 110. Upper lobe 120 may be configured to be received in a cavity defined by a cymba concha and a crus of a helix of the user's ear. For instance, upper lobe 120 may include a front apex portion that, upon a compression an aperture in the upper lobe configured to permit the upper lobe to exhibit a flexion in response to a compression of the upper lobe against the ear to dynamically fit the earpiece to the ear, pushes against an internal surface of the crus of the helix of the ear while upper lobe 120 is disposed in the cavity.

In various embodiments, earpiece 100 may include an aperture 202 in upper lobe 120. Aperture 202 may be configured to permit upper lobe 120 to exhibit a flexion in response to a compression of upper lobe 120 against the user's ear to dynamically fit earpiece 100 to the ear, as discussed in further detail herein.

In various embodiments, earpiece 100 may include an elongate rib 130. Elongate rib 130 may extend between upper end 102 and lower end 104 of arcuate rib 110. In some embodiments, arcuate rib 110 and elongate rib 130 may form a continuous loop defining an interior void 150. In various embodiments, the concha bowl of an ear may be seen through interior void 150 when the user is wearing earpiece 100.

In some embodiments, earpiece 100 may include a monolithic structure (e.g., singular component). In other embodiments, earpiece 100 may include an assembled structure (e.g., a plurality of separate components that have been assembled together to create earpiece 100). For example, arcuate rib 110, upper lobe 120, and elongate rib 130 may include a single continuous molded workpiece. In some embodiments, separate components of earpiece 100 may be attached to each other using various techniques, such as via threads, friction fit, adhesive bonding, ultrasonic welding, or by any other desired method. As understood by one or ordinary skill in the art, earpiece 100 may be manufactured using various techniques, such as additive manufacturing, subtractive manufacturing, molding (e.g., using a pour mold or machine injection molding), and so on. Earpiece 100 may include flexible and/or rigid materials. For instance, earpiece 100 may be composed of various materials, such as polymers (e.g., rubber, plastic, and so on), epoxy, foam, fiberglass, silicone, any combination thereof, and the like. For instance, earpiece 100 may be partially formed of a metal or alloy, such as a metal or alloy comprised of aluminum (anodized aluminum, for example), titanium, magnesium, or steel, a polymer, such as ABS, polycarbonate, or high-density polyethylene, and so on.

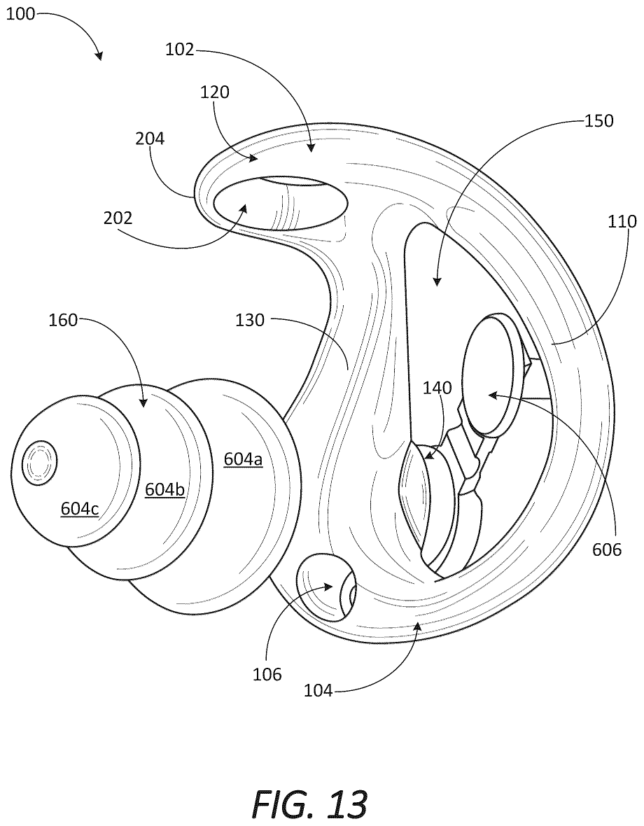

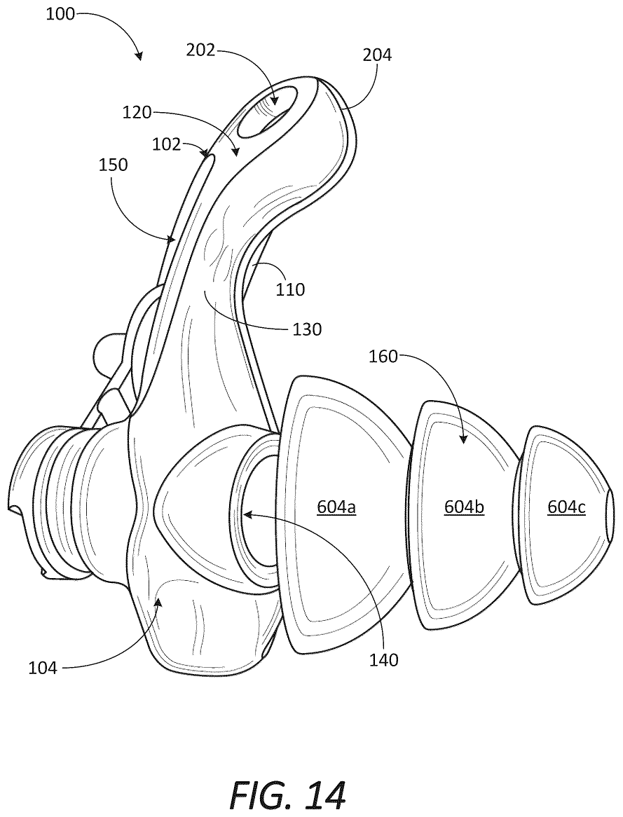

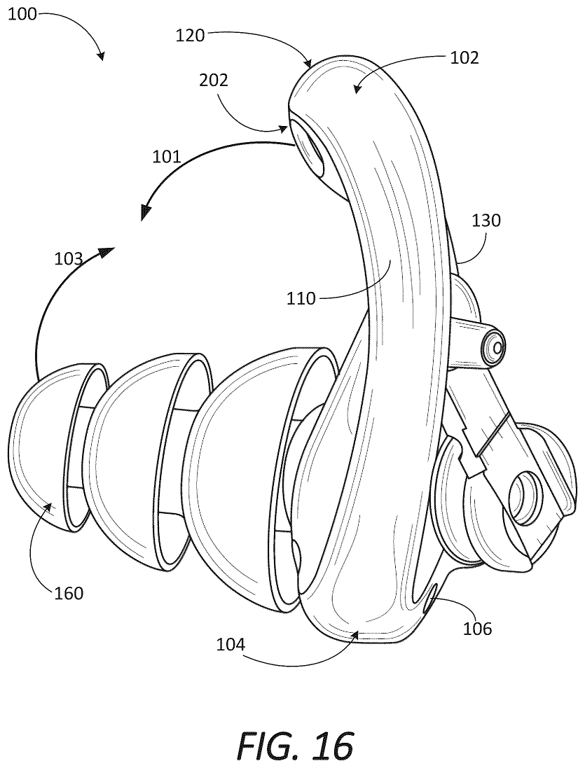

In various embodiments, earpiece 100 may interface with (e.g., connect to) communication equipment. Communication equipment may include mechanical, electrical, or electromechanical components. For instance, communication equipment may include mechanical communication equipment such as an extension (e.g., an extension 160, see FIGS. 13-17) for filtering and/or blocking undesirable sound from reaching the user's eardrum, a buffer (e.g., vibration damping material that mitigates the undesirable transmission of ambient sound and vibration), and so on. Such earpieces (e.g., earplugs) may be suitable for use in gun ranges and noisy industrial environments. In another instance, communication equipment may include cables, circuits, limiting circuits, speakers, microphones, transmitters, transceivers, transducers, and the like (e.g., electrical communication equipment) for transmitting, receiving, and/or filtering sound. In some embodiments, earpiece 100 may be configured to transmit sound using communication equipment (such as incoming cellular telephone transmissions, music, or other sound) to the user's eardrum when disposed within the user's concha. Earpiece 100 may be implemented with any of the features disclosed in U.S. Pat. No. 10,231,048 issued Mar. 12, 2019 and entitled “ERGONOMIC EARPIECE WITH ATTACHMENT MOUNT” and U.S. Pat. No. 10,440,459 issued Oct. 8, 2019 and entitled “ERGONOMIC EARPIECE”, both of which are incorporated by reference herein.

In one or more embodiments, earpiece 100 may include a second aperture 140 configured to receive an extension 160 (see FIGS. 13-16). Extension 160 may traverse through aperture 140 such that, when earpiece 100 is secured to the user's ear, extension 160 extends, at least partially, into the ear canal of the user. In various embodiments, earpiece 100 may include a third aperture 106 configured to receive a lanyard passing therethrough, for example, to hang one or more earpieces 100 from the user's neck. For instance, a first end of the lanyard may connect to a third aperture 106 of a left earpiece 100, and a second end of the lanyard may connect to a third aperture 106 of a right earpiece 100 so as to provide a connection between the separate earpieces 100. This may aid in preventing the misplacement or loss of the earpieces 100 and may allow the user to hang or drape the lanyard around their neck when the earpieces 100 are not in use.

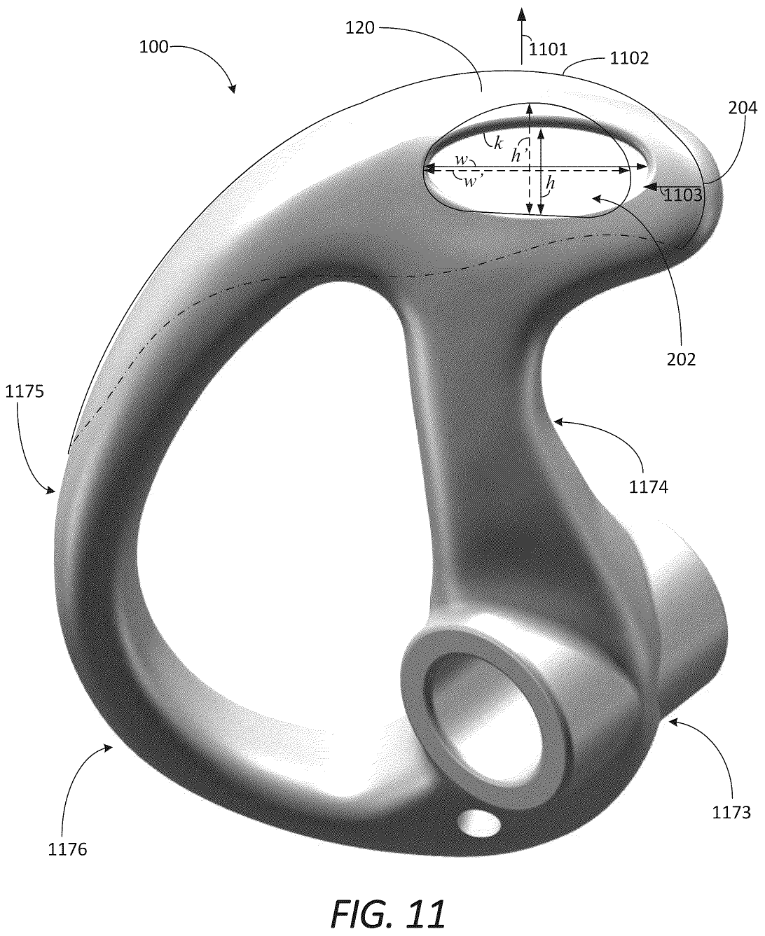

Referring now to FIG. 11, earpiece 100 is illustrated in a rest state and also in a compressed state (denoted by overlayed lines) exhibiting one or more flexions in accordance with an embodiment of the present disclosure. As previously mentioned, earpiece 100 includes upper lobe 120. Upper lobe 120 may include an extended portion of arcuate rib 110, where upper lobe 120 protrudes from arcuate rib 110. Upper lobe 120 may include aperture (e.g., first aperture 202). For instance, first aperture 202 may be at least partially disposed within and, at least partially, defined by upper lobe 120. First aperture 202 may be various shapes and sizes. For example, first aperture 202 may be oval shaped, as shown in FIG. 11 or other shapes as appropriate.

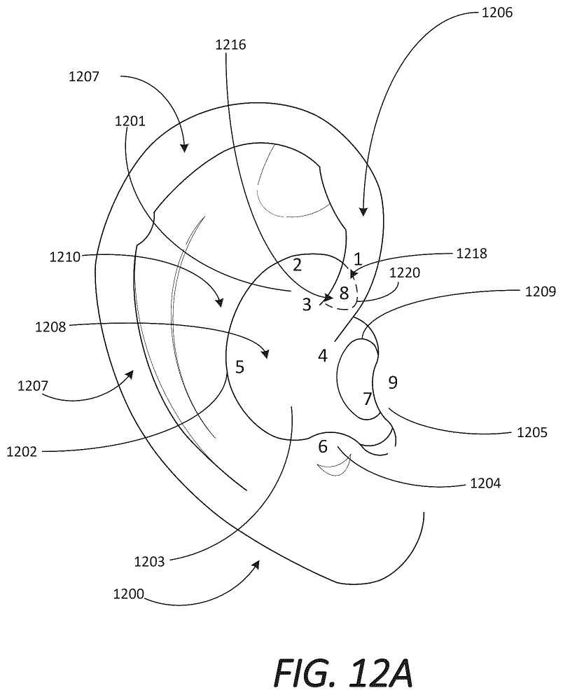

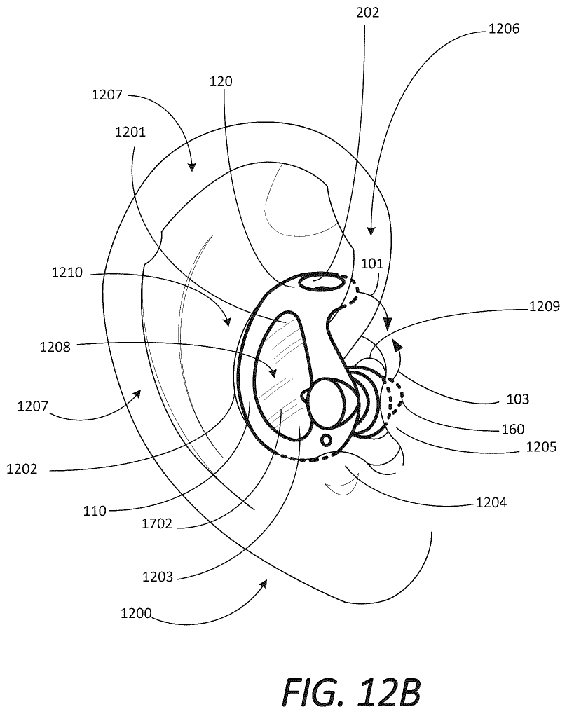

First aperture 202 exhibits a width w and a height h when earpiece 100 is in a rest state (e.g., an initial form while disengaged from the user's ear and in an unsecured position). First aperture 202 exhibits a width w′ and a height h′ when earpiece 100 is in a compressed state (e.g., engaged with the user's ear and in a secured position). Upper lobe 120 extends from upper end 102 of arcuate rib 110 and may be received in a cavity (e.g., cavity 1220, see FIG. 12A) defined by a cymba concha and a crus of a helix of the ear.

First aperture 202 in upper lobe 120 permits upper lobe 120 to exhibit one or more flexions (e.g., first and/or second flexions) in response to a compression of upper lobe 120 against the ear to dynamically fit earpiece 100 to the ear. A first flexion in a first direction 1101 may result in a top portion 1102 of upper lobe 120 biasing at least a portion of the antihelix of the ear. For example, the first flexion may include expansion of top portion 1102 of upper lobe 120 into a second cavity 1218, which is defined by cymba concha 1201 and concha bowl ridge 1202 of ear 1200, to deform upper lobe 120 to complement second cavity 1218 to fit earpiece 100 in ear 1200 (see FIG. 12A). In another example, a second flexion in a second direction 1103 includes a deformation of upper lobe 120 to complement cavity 1220 (e.g., first cavity 1216) to fit earpiece 100 in ear 1200 (see FIG. 12A).

The flexions exhibited by earpiece 100, as shown by the overlayed lines in FIG. 11, may include a deformation of upper lobe 120 and/or aperture 202. For example, and without limitation, height h may change (e.g., increase) to height h′ during the first flexion of upper lobe 120 in first direction 1101. In some embodiments, height h′ may be greater than height h due to a displacement of top portion 1102 and/or a bottom portion 1110 of upper lobe 120 during the first flexion. For instance, the cavity that receives upper lobe 120 may include a first cavity 1216 (see FIG. 12A), and the first flexion includes an expansion of top portion 1102 of upper lobe 120 into a second cavity 1218 (see FIG. 12A) defined by the cymba concha and a concha bowl ridge of the ear to deform upper lobe 120 to complement the second cavity to fit earpiece 100 in the ear.

In one or more embodiments, the first flexion includes a deformation of upper lobe 120 to complement the cavity to fit the earpiece in the ear. In some embodiments, the first flexion may include a second displacement of bottom portion 1110 in a third direction 1104. Thus, aperture 202 may increase in height (e.g., from height h to height h′) by an expansion in one or both directions 1101 and/or 1104.

In another example, and without limitation, width w may change to width w′ during the second flexion of upper lobe 120 in second direction 1103. In one or more embodiments, during the second flexion, the width w of aperture 202 may decrease. In various embodiments, top portion of upper lobe 120 may bend, for example, upward during the second flexion. For instance, upper lobe 120 may bend (e.g., a curvature k of aperture 202 may deform to a curvature k′) in response to the second flexion. For example, the compression of earpiece 100 may push front apex portion 204 of upper lobe 120 against an internal surface of the crus of the helix of the ear while upper lobe 120 is disposed in the cavity. Thus, front apex portion 204 of upper lobe 120 may bias an internal surface of the helical crus (e.g., crus of the helix) while upper lobe 120 is disposed within the cavity of the user's ear such that a force is exerted on front apex portion 204 by the internal surface in direction 1103. The force exerted on front apex portion 204 causes compression of upper lobe 120, which results in first aperture 202 being compressed. The compression of upper lobe 120 causes the width of aperture 202 to change from width w to width w′ (e.g., flexion width) and/or the height of aperture 202 to change from height h to height h′ (e.g., flexion height). Accordingly, it will be appreciated that, in some embodiments, the first and second flexions may occur simultaneously in response to compression of front apex portion 204 of upper lobe 120 against the internal surface of the crus of the helix of the ear.

Referring now to FIG. 12A, grab points 1-9 for earpiece 100 on an ear 1200 are shown in accordance with an embodiment of the present disclosure. In various embodiments, earpiece 100 may include a substantially D-shaped configuration. Earpiece 100 may include arcuate rib 110 that has upper and lower ends 102 and 104, respectively. In some embodiments, arcuate rib 110 may be attached to elongate rib 130 at, for example, upper and lower ends 102, 104 of arcuate rib 110. Upper lobe 120 may be formed proximate where arcuate rib 110 and elongate rib 130 join at the top of earpiece 100. For example, upper lobe 120 (e.g., top lobe) may be formed at the top of earpiece 100, near where arcuate rib 110 and elongate rib 130 intersect. A smooth curve may be formed proximate where arcuate rib 110 and elongate rib 130 join at the bottom of earpiece 100, which, in some embodiments, may engage a tragus 1205 and antitragus 1204 of ear 1200.

As shown in FIG. 12A, a (right) human ear 1200 (e.g., such as the outer portion of the ear or pinna) typically has anatomical structures such as a cymba concha 1201, a concha bowl 1208, a ridge 1202 of concha bowl 1208, a cavum concha 1203, an antitragus 1204, a tragus 1205, a helix 1207, a crus 1206 of helix 1207, and an antihelix 1210.

According to an embodiment, examples of grab points 1-9 are provided by at least some of the various anatomical structures of ear 1200. The grab points 1-9 and/or anatomical structures of the ear proximate the grab points 1-9 can grab or otherwise hold, capture, and/or maintain earpiece 100 (see FIG. 12B, for example) within ear 1200, e.g., within the concha bowl 1208 thereof. For example, the grab points 1-9 can define detents, grabbers, locks, fingers, tabs, or other structures or features that engage the earpiece 100 and mechanically limit undesirable movement of the earpiece 100.

Thus, the grab points 1-9 can tend to resist movement of earpiece 100. More particularly, the grab points 1-9 can tend to resist movement of earpiece 100 that would result in separation of earpiece 100 from ear 1200. The grab points 1-9 can capture earpiece 100 substantially within concha bowl 1208. The grab points 1-9 can tend to resist other movement of earpiece 100. For example, the grab points can tend to resist translational or rotational movement of earpiece 100 that would move earpiece 100 away from a desired position substantially within concha bowl 1208. Earpiece 100 can be configured to complement a portion of ear 1200, so as to facilitate the use of the grab points 1-9. For example, earpiece 100 can be substantially a mirror image of one or more portions of ear 1200.

According to an embodiment, a first grab point 1 can be at a forward and uppermost end of the cymba concha 1201. Front apex portion 204 (see FIG. 11) of earpiece 100 can be disposed underneath the ridge of cymba concha 1201 (e.g., beneath the inferior crus) and behind the helix 1207 just above the crus 1206 of helix 1207.

According to an embodiment, a second grab point 2 can be located where a top portion 1102 of earpiece 100 fits snugly under or puts pressure against a top part of the ridge of the concha bowl 1208 (e.g., beneath the inferior crus).

According to an embodiment, a third grab point 3 can be an underneath lower portion of earpiece 100 when earpiece 100 is placed within concha bowl 1208. The lower portion 1173 of earpiece 100 can be captured beneath crus 1206 of helix 1207.

According to an embodiment, a middle front part 1174 (see FIG. 11) of earpiece 100 may go over the crus 1206 of helix 1207 and slightly into ear canal 1209 to define a fourth grab point 4 on top of and underneath crus 1206 of helix 1207.

According to an embodiment, a fifth grab point 5 may extend from where a top part of the cavum concha 1203 splits into the narrow flexible ring that forms itself to the backside of the concha bowl 1208. The fifth grab point 5 may grab the back side 1175 (see FIG. 11) of earpiece 100.

According to an embodiment, a sixth grab point 6 may be defined at the bottom of ear 1200. The sixth grab point 6 may grab earpiece 100 at the flare 1176 (see FIG. 11) of earpiece 100. The flare 1176 may continue around to the underside of the antitragus 1204 and the rim of the outer portion of ear canal 1209.

According to an embodiment, a seventh grab point 7 may be defined by an underside of tragus 1205. The seventh grab point 7 may grab lower portion 1173 of earpiece 100.

According to an embodiment, an eighth grab point 8 may be defined by a cavity defined by cymba concha 1201 and crus 1206 of helix 1207 when arcuate rib 110 is received by antihelix 1210 and antitragus 1204 of ear 1200. More specifically, at least a portion of upper lobe 120 may be received (as shown by the broken lines of upper lobe 120 in FIG. 12B) in cavity 1220 defined by cymba concha 1201 and a crus 1206 and bias downward in direction 101 (see FIG. 12B) within the cavity to create grab point 8.

In one or more embodiments, cavity 1220 is a first cavity 1216 and a second cavity 1218. As previously mentioned, the flexion may include the first flexion and the second flexion. First cavity 1216 be defined by cymba concha 1201 and crus 1206. In various embodiments, the first flexion includes expansion of top portion 1102 (see FIG. 11) of upper lobe 120 into second cavity 1218, which is defined by cymba concha 1201 and concha bowl ridge 1202 of ear 1200, to deform upper lobe 120 to complement second cavity 1218 to fit earpiece 100 in ear 1200. In one or more embodiments, the second flexion includes a deformation of upper lobe 120 to complement cavity 1220 (e.g., first cavity 1216) to fit earpiece 100 in ear 1200.

According to an embodiment, a ninth grab point 9 may be defined by ear canal 1209. Extension 160 of earpiece 100 may be bent such that extension at least partially biases a surface of ear canal 1209. For example, extension 160 may apply pressure to a top of ear canal 1209, as indicated by direction 103 (see FIG. 12B). In other some embodiments, extension 160 may apply pressure to all of ear canal 1209 so as to seal the ear canal 1209 and thus provide sound attenuation. In various embodiments, grab points 8 and 9 may work in tandem to secure earpiece 100 to ear 1200. For instance, upper lobe 120 and extension 160 may be biased toward each other, in directions 101 and 103, respectively, forming a pair of clamp jaws to secure earpiece 100 in ear 100.

According to various embodiments, additional grab points may be provided. The configuration and size of the anatomical structures and features of the human ear can vary substantially from one individual to another. Not all of the described grab points will necessarily limit the motion of earpiece 100 in every instance. In some ears, only a portion of the grab points will limit the motion of earpiece 100. Other anatomical structures or features of the ear can function as grab points. Thus, the discussion and illustration of grab points is by way of example only, and not by way of limitation.

Now referring to FIG. 12B, earpiece 100 disposed within ear 1200 is shown in accordance with an embodiment of the present disclosure. Earpiece 100 may include an extension 160, arcuate rib 110, elongate rib 130, and upper lobe 120 with first aperture 202, as previously discussed herein.

In some embodiments, earpiece may include a membrane 1702. Membrane 1702 disposed across interior void 150. Membrane 1702 may be configured to attenuate sound passing from an external environment to interior void 150. Membrane may provide a barrier that prevents sound from reaching ear canal 1209. Membrane 1702 may be formed of a flexible and/or rigid material. For instance, membrane 1702 may be composed of a polymer (e.g., rubber, plastic, or the like), fiberglass, silicone, and the like. In a non-limiting embodiments, membrane 1702 may be formed form a flexible material to attenuate sound while still allowing arcuate rib 110 to flex.

As previously mentioned in FIG. 12A, eighth grab point 8 may be defined by cavity 1220 defined by cymba concha 1201 and crus 1206. Thus, at least a portion of upper lobe 120 may be received in cavity 1220 and bias downward in direction 101 within cavity 1220 to create grab point 8. Furthermore, ninth grab point 9 may be defined by ear canal 1209, where extension 160 of earpiece 100 may be bent such that extension at least partially biases a surface of ear canal 1209 (e.g., top of ear canal 1209). In various embodiments, grab points 8 and 9 may work in tandem to comfortably secure earpiece 100 to ear 1200. For instance, upper lobe 120 and extension 160 may be biased toward each other, in directions 101 and 103, respectively, forming a pair of clamp jaws to secure earpiece 100 in ear 100.

In an exemplary embodiment, earpiece 100 may include arcuate rib 110 having upper and lower ends, elongate rib 130 extending substantially between the upper and lower ends, and upper lobe 120 formed at the upper end of arcuate rib 110. A smooth rounded surface can be formed at the lower end of arcuate rib 110. Earpiece 100 can be configured to be generally shaped like a “D”. However, those of ordinary skill in the art will appreciate that other embodiments are likewise suitable. For example, either elongate rib 130 or arcuate rib 110 may be modified substantially or omitted.

In some embodiments, when earpiece 100 is used in an ear where arcuate rib 110 is too large for the ear (e.g., does not fit within the concha bowl without substantial deformation), arcuate rib 110 may merely bend or collapse without adverse effect. In various exemplary embodiments, the arcuate rib 110 may bend to accommodate a wide range of ear sizes. For example, the arcuate rib 110 can collapse so as to accommodate a range of ear sizes. Earpiece 100 may be formed of a resilient polymer, allowing earpiece 100 to bend as needed. For example, in one embodiment, earpiece 100 may be formed of a resilient polymer having a Shore A durometer of between 35 and 45. More particularly, earpiece 100 may be formed of a resilient polymer having a Shore A durometer of approximately 40. Alternatively, in another embodiment, earpiece 100 may be formed of a more rigid material. Earpiece 100 may be configured to fit a majority of ear sizes, which may be facilitated by the bending of arcuate rib 110 and deformation of upper lobe 120.

According to an embodiment, earpiece 100 is configured to fit multiple sizes of ears. More particularly, arcuate rib 110 and upper lobe 120 are each deformable so as to permit earpiece 100 to fit into various sized concha bowls, such as smaller concha bowls.

In various embodiments, earpiece 100 may be configured to be disposed and held in place within a concha bowl 1208 of a human ear 1200. More particularly, a helical crus 1206 of the helix 1207 and the antihelix 1210 of a wearer's ear may cooperate to capture upper lobe 120. Antihelix 1210 and antitragus 1204 may cooperate to capture arcuate rib 110. Thus, earpiece 100 is configured to be captured by protrusions of concha bowl 1208. In this manner, earpiece 100 is held firmly in place within concha bowl 1208 and can therefore maintain extension 160 (see FIGS. 13-16) in a desired position within ear canal 1209.

Referring now to FIGS. 13-16, various views of earpiece 100 having extension 160 are shown in accordance with embodiments of the present disclosure. One exemplary embodiment includes earpiece 100 having extension 160 (also referred to as an “insert” and an “ear insert”) attached thereto. In some embodiments, earpiece 100 is configured to be disposed in concha bowl 1208 of outer ear 1200 (see FIG. 12A). Extension 160 may be configured to be disposed within ear canal 1209 (see FIG. 12B).

As previously mentioned, earpiece 100 is held in place by anatomical structures of the user's ear. In some embodiments, earpiece 100 holds the extension 160 of earpiece 100 in place within ear canal 1209 of the user's ear 1200 (see FIG. 12B). That is, earpiece 100 prevents extension 160 from loosening or falling out of ear canal 1209. According to one embodiment, earpiece 100 positions a sound port at the distal end of extension 160 near the eardrum of ear 1200 so that the volume of a two-way radio can be reduced. That is, earpiece 100 determines how far into the ear extension extends 160. In various embodiments, extension 160 may be configured so as to prevent loud, annoying, distracting, and/or harmful sounds from reaching the eardrum of the user. In some embodiments, extension 160 may provide noise-cancelling capabilities, such as electronics that provide noise-cancelling signals or mechanical components that obstruct sound from reaching the eardrum.

In various embodiments, extension 160 may include a stem 608. Stem 608 may include a cylindrical stem extending from a lower portion 1173 (see FIG. 11) of earpiece 100. In some embodiments, stem 608 may be solid. In other embodiments, stem 608 may be hollow (e.g., a tubular stem having a bore). Stem 608 may be configured to transverse (e.g., extend) through at least a portion of second aperture 140. Various combinations of sound attenuation and sound transmission may be provided by earpiece 100. For example, a hollow or partially hollow extension 160 may be configured so as to substantially attenuate some ambient sound (such as potentially harmful loud noise), while allowing some ambient sound (such as voices) to be heard. Optionally, extension 160 may include one or more openings that allow a substantial portion of ambient sound to be heard, while also allowing radio communications to be heard. Optionally, a filter may be used to selectively allow sounds to be heard when earpiece 100 is disposed with concha bowl 1208 of ear 1200.

Extension 160 may pass though aperture 140 of earpiece 100 and extend into at least a portion of ear canal 1209 of the user while earpiece 100 is secured to ear 1200 of the user to pass audio communication to the user and/or to attenuate the external noises. In one or more embodiments, stem 608 may extend away from elongate rib 130 and may be configured to enter ear canal 1209. In an embodiment, stem 608 may include a bore, where the bore formed within extension 160 transmits sound to a point proximate the eardrum. Acoustic tubing attached to earpiece 100 such that a contiguous sound channel may be formed by the acoustic tubing and bore of stem 608. The bore may transmit sound, such as from a two-way radio, to the wearer's eardrum.

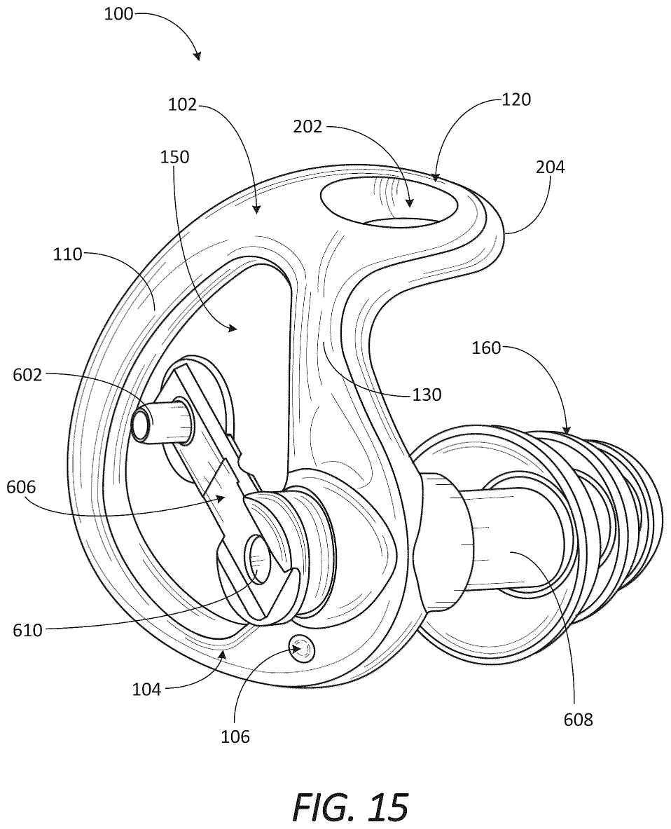

Earpiece 100 may also be used as a sound attenuating earplug, where extension 160 may be inserted into ear canal 1209 and prevent sound from reaching the user's eardrum (e.g., attenuate external noises received by the ear of the user). Alternatively, a member that seals or partially seals the ear can be added to the earpiece 100. For instance, earpiece 100 may include a cap 606 that may be attached to stem 608 and may be selectively transitioned between an open position (see FIG. 15), to pass the external noises to the user's ear canal, and a closed position (see FIG. 12B), to attenuate the external noises. For example, cap 606 may include a protrusion 602 configured to be received by a hole 610 of cap 606 when cap 606 is in the closed position. For example, protrusion 602 may extend from a surface of a lid of cap 606 that may be pivoted (e.g., using a bendable or hinged component of cap 606) until protrusion 602 is disposed within hole 610. In some embodiments, hole 610 of cap 606 may be in fluidic communication with the bore of extension 160 so that, when cap 606 is in the open position, external noise may pass through the hole and bore to the eardrum.

In various embodiments, extension 118 may have three flanges 604a, 604b, and 604c formed upon stem 608 thereof (see FIG. 15). In some embodiments, flanges 604a, 604b, and 604c may be dome-shaped and radially extend from stem 608. As those skilled in the art will appreciate, the use of more flanges generally provides better sound reduction. The use of more flanges can also better secure extension 160 within ear canal 1209. Although three flanges are shown, extension 160 may include more or less flanges. For example, extension 160 may include three, four, five, six, or more flanges, or no flanges. The flanges 604a, 604b, and 604c need not be identical, but rather may vary in size, shape, orientation and/or positions of attachment to stem 608, for example.

According to various embodiments, earpiece 100 may include a flanged extension that is suitable for use as hearing protection, is suitable for facilitating listening to a two-way radio or the like, or is suitable for providing a combination of hearing protection and such listening, as previously mentioned herein. One or more embodiments may include a solid extension so as to substantially attenuate ambient sound. Alternatively, one or more embodiments can comprise a hollow or partially hollow extension to allow at least some ambient sound or sound from a radio to pass therethrough.

In one or more embodiments, extension 160 may be either removably or permanently attached to earpiece 100. For instance, extension 160 may be removably attached to earpiece 100 by friction fit, by detents, by threads, or by any other desired means. For example, extension 160 may be friction fit to earpiece 100 by sizing a proximal portion of extension 160 so as to fit tightly within aperture 140 of earpiece 100. Extension 160 may be permanently attached to earpiece 100 by adhesive bonding, ultrasonic welding, or by any other desired means. Alternatively, extension 160 may be integrally formed to earpiece 100, such as by injection molding earpiece 100 and extension 160 within a common mold cavity. Thus, earpiece 100 and extension 160 can be formed either integrally or separately.

Earpiece 100 and extension 160 may be formed of a soft, resilient material to enhance comfort during use. Both earpiece 100 and extension 160 may be formed of the same material. For example, earpiece 100 and extension 160 may be formed of a resilient polymer, such as silicon rubber. For example, in one embodiment, earpiece 100 and extension 160 may be formed of a material having a Shore A durometer of between 35 and 45, such as a Shore A durometer of approximately 40. Alternatively, in another embodiment, the earpiece 100 can be formed of a more rigid material.

In one embodiment, earpiece 100 may function as a stop to prevent extension 160 from being inserted too far into the ear. In one embodiment, earpiece 100 may also prevent extension 160 from being inadvertently removed or loosened from the ear of the user. In one embodiment, the length of extension 160 may determine, at least in part, how close the tip thereof is positioned with respect to the eardrum.

Extension 160 may include any desired number of flanges, including no flanges at all. For example, stem 608 may include a flexible material (e.g., foam or silicone) that may be deformed to be inserted into the ear canal. Stem 608 may either be solid (so as to substantially block sound) or hollow (so as to substantially transmit sound), as previously mentioned herein. Stem 608 may be partially hollow, so as to selectively transmit sound.

In some embodiments, stem 608 may be substantially straight. In other embodiments, stem 608 may be curved or angled. For instance, stem 608 may bend such that stem 608 angles upwardly to conform to the upward angle of the ear canal 1209. For example, in one embodiment, stem 608 may bend such that it angles upwardly at an angle, angle A, of approximately 30°. In one embodiment, the distance between the proximal end of extension 160 and the distal end of outer flange 604a, dimension B, can be approximately 0.545 inch. In one embodiment, the distance between the proximal end of extension 160 and the point where a filter (such as a Hocks filter) ends, dimension C, can be approximately 0.304 inch. In one embodiment, the distance between the proximal end of extension 160 and the bend in stem 608, dimension D, can be approximately 0.680 inch. In one embodiment, the distance between the proximal end of extension 160 and a distal end of inner flange 604c, dimension E, can be approximately 0.743 inch. In one embodiment, the distance between the proximal end of extension 160 and the distal end of inner flange 604c, dimension F, can be approximately 0.870 inch. Bore and/or openings in stem 608 and/or flanges 604a, 604b, and 604c can be configured so as to selectively transmit and block desired sounds. Such selectivity can be based upon the frequency and/or intensity of the sound.

In one embodiment, the diameter of stem 608 can be approximately 0.189 inch. Stem 608 can optionally have a bore formed therethrough. In one embodiment, bore may include a diameter of approximately 0.094 inch. In one embodiment, outer flange 604a may include a radius of approximately 0.241 inch. Similarly, in one embodiment, inner flange 604c may include a radius of approximately 0.193 inch. Thus, the radius of inner flange 604c can be substantially less than the radius of outer flange 604a, so as to better accommodate the manner in which the ear canal 1209 becomes narrower as it gets deeper (e.g., closer to the eardrum).

The exemplary angles and dimensions discussed above provide a single extension 160 that is suitable for use with a large number of individuals. Those skilled in the art will appreciate that other dimensions are likewise suitable.

Petitions, baffles, and/or restrictions (such as portions of reduced diameter), as well as openings in the stem and/or flanges, can be configured so as to modify sound transmitted through extension 160 in a desired manner. For example, sound within the voice range of frequencies can be selectively passed through the extension with comparatively less attenuation and sounds outside of the voice range can be selectively attenuated. In some embodiments, extension 160 may be configured so as to lack a bore. Thus, the user can wear one earpiece having an extension with a bore and one earpiece lacking a bore. The earpiece having an extension with a bore facilitates listening to a radio, while the earpiece having an extension without a bore at least partially blocks distracting and/or potentially harmful ambient sound (it functions as an earplug). In a similar manner, one earpiece can be configured so as to selectively pass voice and to selectively mitigate other sounds. The other earpiece can be configured so as to selectively pass all of the sound from a two-way radio. In various embodiments, a filter can be inserted into the bore of extension 160 to selectively mitigate sound exposure. For example, a Hocks filter can be used to mitigate exposure to louder sounds, while still allowing a wearer to hear quieter sounds, such as speech.

In some embodiments, extension 160 includes one or more flanges having a skin or covering formed thereover. Covering can be formed of a thin resilient material, such as rubber, such as that of which common balloons are formed. Optionally, foam or gel can be disposed between the covering and flanges. Foam or gel may include a biocompatible material, such as a silicon.

In certain embodiments, any of the flanges disclosed herein may be formed integrally with the stem of an associated extension. In other embodiments, the flanges may be formed separate from the stem and may be formed of a different material with respect thereto.

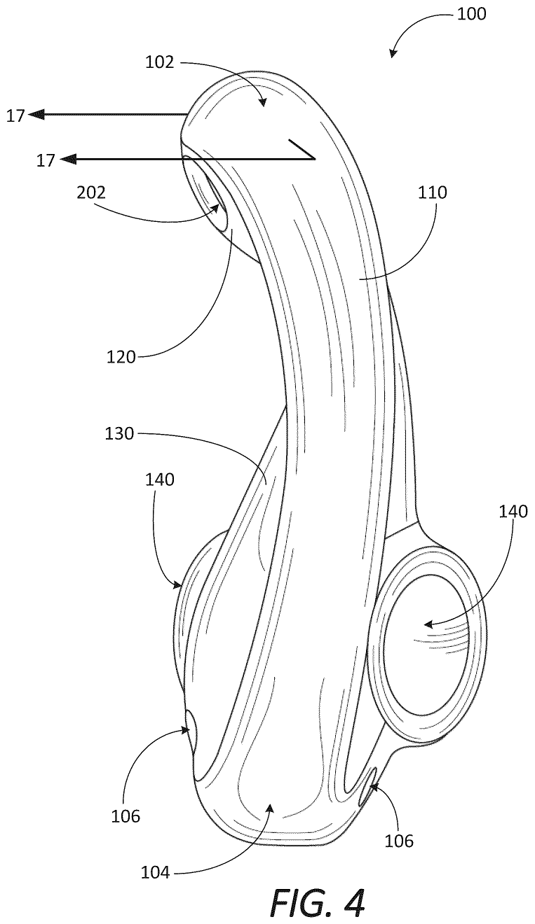

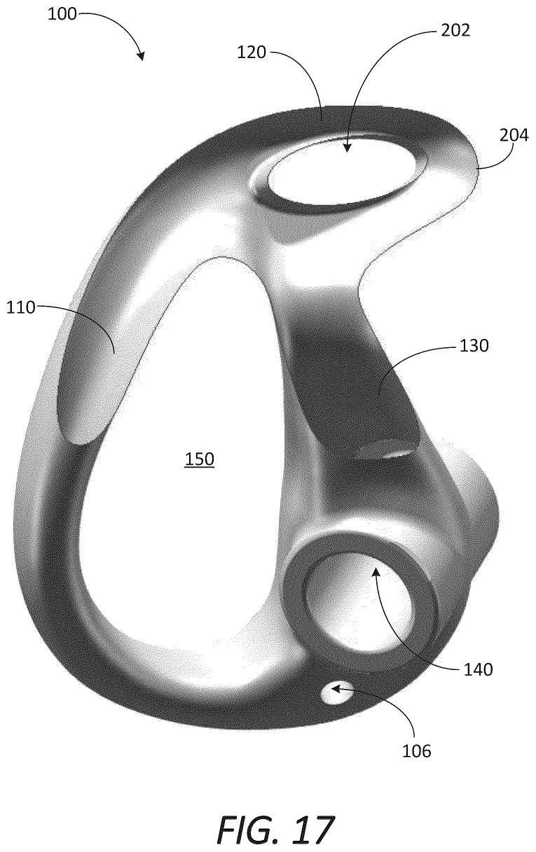

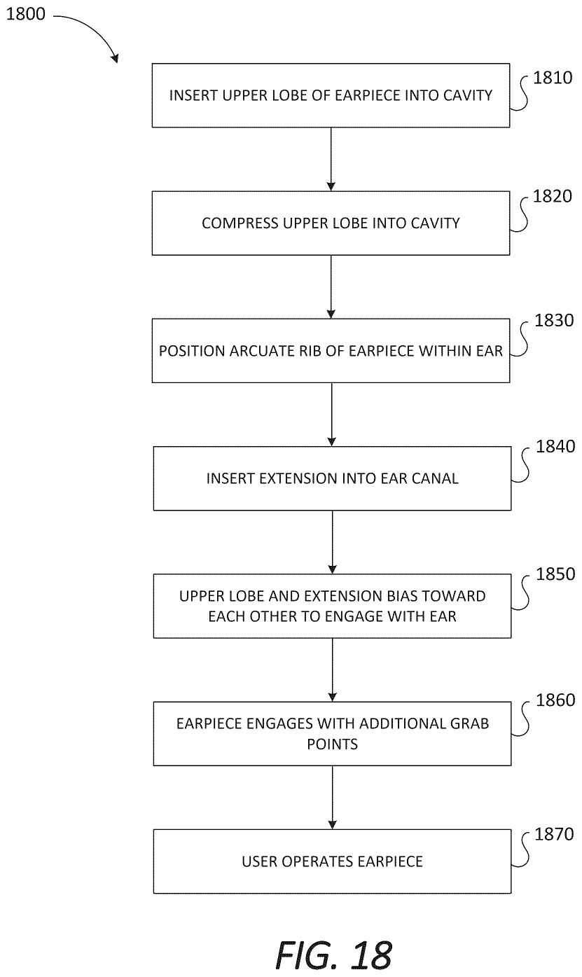

FIG. 17 illustrates a cross-sectional view of the earpiece of FIG. 4, as seen along the lines of the section 17-17 taken therein, in accordance with an embodiment of the present disclosure. In various embodiments, at least a portion of arcuate rib 110 and/or at least a portion of elongate rib 130 may be substantially hollow. Earpiece 100 being hollow may reduce weight of earpiece 100 and provide further flexibility of earpiece 100 or components thereof. As shown in FIG. 18, in some embodiments, earpiece 100 may be entirely hollow. In other embodiments, earpiece 100 may be solid. In other embodiments, earpiece may include solid and hollow portions.

FIG. 18 illustrates a flow chart of a process 1800 of securing an earpiece to an ear of a user in accordance with an embodiment of the present disclosure. For explanatory purposes, process 1800 is primarily described within this disclosure with reference to earpiece 100 and its associated arrangement of components as described in FIGS. 1-18. However, process 1800 is not limited to such implementations. Any step, sub-step, sub-process, or block of process 1800 may be performed in an order or arrangement different from the embodiments illustrated in FIG. 18; some may be omitted, others may be added, and some may be performed simultaneously as appropriate.

A shown at block 1810, process 1800 includes inserting upper lobe 120 of earpiece 100 into a cavity. For instance, process 1800 may include inserting upper lobe 120 into a cavity defined by a cymba concha and a crus of a helix of the ear. In some embodiments, earpiece further includes elongate rib 130, which extends between upper and lower ends 102 and 104 of arcuate rib 110. Arcuate rib 110 and elongate rib 130 may form a continuous loop defining an interior void 150. In some embodiments, earpiece 100 may include a membrane 1702 disposed across interior void 150. Process 1800 may include attenuating, by membrane 1702, sound passing from an external environment to interior void 150. In various embodiments, arcuate rib 110, upper lobe 120, and elongate rib 130 include a single continuous molded workpiece. Interior void 150 may be defined by the single continuous molded workpiece. In some embodiments, at least a portion of arcuate rib 110 and/or at least a portion of elongate rib 130 is substantially hollow, as discussed in FIG. 18.

As shown at block 1820, process 1800 includes compressing upper lobe into the cavity. For example, process 1800 may include compressing upper lobe 120 against the ear to exhibit a flexion to dynamically fit the earpiece to the ear. In one or more embodiments, the compressing pushes front apex portion 204 of upper lobe 120 against an internal surface of the crus of the helix of the ear while upper lobe 120 is disposed in the cavity. The cavity may include a first cavity, and the flexion may include a first flexion. The first flexion may include an expansion of top portion 1102 of upper lobe 120 into a second cavity defined by the cymba concha and a concha bowl ridge of the ear to deform upper lobe 120 to complement the second cavity to fit earpiece 100 in the ear. In some embodiments, the flexion may include a second flexion, where the second flexion includes a deformation of upper lobe 120 to complement the cavity to fit earpiece 100 in the ear.

As shown at block 1830, process 1800 includes positioning arcuate rib 110 of earpiece 100 within the user's ear. For instance, process 1800 may include positioning arcuate rib 110 against an antihelix and an antitragus of the ear.

As shown at block 1840, process 1800 includes inserting extension 160 into the ear canal of the user's ear. In some embodiments, aperture 202 is a first aperture, and earpiece 100 further includes a second aperture 140 disposed in at least one of arcuate rib 110 and/or elongate rib 130 that is configured to receive extension 160 passing therethrough. In one or more embodiments, process 1800 includes inserting at least a portion of extension 160 into an ear canal of the ear. In one or more embodiments, earpiece 100 further includes extension 160. Upper lobe 120 and extension 160 may be biased toward each other forming a pair of clamp jaws. In various embodiments, process 1800 includes clamping earpiece 100 against the ear by the clamp jaws to secure earpiece 100 in the ear.

As shown at block 1850, process 1800 includes upper lobe and extension biasing toward each other to engage with the ear.

As shown at block 1860, process 1800 includes earpiece 100 engaging with additional grab points.

As shown at block 1870, process 1800 includes the user operating earpiece 100. Operating earpiece 100 may include the various methods and techniques of operating earpiece 100 previously discussed in this disclosure. One or more embodiments can mitigate noise exposure and/or facilitate communications. For instance, operating earpiece may include using earpiece 100 for noise-cancelling purposes or to attenuate sound. Noise exposure is mitigated by at least partially blocking the ear canal with extension 160 from earpiece 100. Communications may be facilitated by providing a passage or bore for sound through extension 160, where the bore may be covered by a cap for selective facilitation of communication. In another instance, operating earpiece 100 may include using earpiece for one or two-way communication. For example, operating earpiece 100 may include using earpiece 100 for two-way communication, hearing protection, and so on. In various embodiments, extension 160 may be a portion of a communications cable, a wireless telecommunications device (e.g., a wireless audio headphone or a Bluetooth® headset with headphone and microphone), an audio headphone (e.g., an earbud), an earplug, or other type of device configured to mate with earpiece 100. Extension 160 extends to a point proximate the eardrum so that sound is delivered more directly to the eardrum. Thus, less volume is needed. The use of less volume is useful in covert operations. As stated above, it may also facilitate the use of smaller, less powerful, and/or less expensive speakers.

One or more embodiments of the present invention provide an earpiece that is more comfortable, less costly, easier to inventory, and more effective with respect to contemporary earpieces. Although described herein as being for use in human ears, one or more embodiments can also be used in non-human ears. For example, an embodiment can be configured for canine ears, to mitigate noise exposure and/or facilitate communication with police or military dogs. As those skilled in the art will appreciate, such dogs are commonly exposed to noisy environments, such as those environments sometimes encountered in police work and on the battlefield. Further, it is frequently desirable to communicate with such dogs. Their ability to respond to radio commands has been established.

Where applicable, various embodiments provided by the present disclosure can be implemented using hardware, software, or combinations of hardware and software. Also, where applicable, the various hardware components and/or software components set forth herein can be combined into composite components comprising software, hardware, and/or both without departing from the spirit of the present disclosure. Where applicable, the various hardware components and/or software components set forth herein can be separated into sub-components comprising software, hardware, or both without departing from the spirit of the present disclosure. In addition, where applicable, it is contemplated that software components can be implemented as hardware components, and vice versa.

Software in accordance with the present disclosure, such as non-transitory instructions, program code, and/or data, can be stored on one or more non-transitory machine-readable mediums. It is also contemplated that software identified herein can be implemented using one or more general purpose or specific purpose computers and/or computer systems, networked and/or otherwise. Where applicable, the ordering of various steps described herein can be changed, combined into composite steps, and/or separated into sub-steps to provide features described herein.

The foregoing description is not intended to limit the present disclosure to the precise forms or particular fields of use disclosed. Embodiments described above illustrate but do not limit the invention. It is contemplated that various alternate embodiments and/or modifications to the present invention, whether explicitly described or implied herein, are possible in light of the disclosure. Accordingly, the scope of the invention is defined only by the following claims.

What is claimed is:

1. An earpiece comprising:
an arcuate rib having upper and lower ends, wherein the arcuate rib is configured to be received by an antihelix and an antitragus of an ear;
an upper lobe extending from the upper end of the arcuate rib and configured to be received in a cavity defined by a cymba concha and a crus of a helix of the ear; and
an aperture in the upper lobe configured to permit the upper lobe to exhibit a flexion in response to a compression of the upper lobe against the ear to dynamically fit the earpiece to the ear.

2. The earpiece of claim 1, wherein the compression pushes a front apex portion of the upper lobe against an internal surface of the crus of the helix of the ear while the upper lobe is disposed in the cavity.

3. The earpiece of claim 2, wherein:
the cavity is a first cavity; and
the flexion is a first flexion comprising an expansion of a top portion of the upper lobe into a second cavity defined by the cymba concha and a concha bowl ridge of the ear to deform the upper lobe to complement the second cavity to fit the earpiece in the ear.

4. The earpiece of claim 2, wherein the flexion is a second flexion comprising a deformation of the upper lobe to complement the cavity to fit the earpiece in the ear.

5. The earpiece of claim 1, further comprising an elongate rib extending between the upper and lower ends of the arcuate rib.

6. The earpiece of claim 5, wherein:
the aperture is a first aperture;
the earpiece further comprises a second aperture disposed in at least one of the arcuate rib and/or the elongate rib and configured to receive an extension passing therethrough; and
at least a portion of the extension is configured to be received by an ear canal of the ear.

7. The earpiece of claim 6, further comprising:
the extension; and
wherein the upper lobe and the extension are biased toward each other forming a pair of clamp jaws to secure the earpiece in the ear.

8. The earpiece of claim 5, wherein:
the arcuate rib and the elongate rib form a continuous loop defining an interior void; and
the earpiece further comprises a membrane disposed across the interior void and configured to attenuate sound passing from an external environment to the interior void.

9. The earpiece of claim 5, wherein the arcuate rib, the upper lobe, and the elongate rib comprise a single continuous molded workpiece.

10. The earpiece of claim 5, wherein at least a portion of the arcuate rib and/or at least a portion of the elongate rib is substantially hollow.

11. A method comprising:
securing an earpiece to an ear, wherein the earpiece comprises:
an arcuate rib having upper and lower ends,
an upper lobe extending from the upper end of the arcuate rib, and
an aperture in the upper lobe; and
wherein the securing comprises:
inserting the upper lobe into a cavity defined by a cymba concha and a crus of a helix of the ear,
compressing the upper lobe against the ear to exhibit a flexion to dynamically fit the earpiece to the ear, and
positioning the arcuate rib against an antihelix and an antitragus of the ear.

12. The method of claim 11, wherein the compressing pushes a front apex portion of the upper lobe against an internal surface of the crus of the helix of the ear while the upper lobe is disposed in the cavity.

13. The method of claim 12, wherein:
the cavity is a first cavity; and
the flexion is a first flexion comprising an expansion of a top portion of the upper lobe into a second cavity defined by the cymba concha and a concha bowl ridge of the ear to deform the upper lobe to complement the second cavity to fit the earpiece in the ear.

14. The method of claim 12, wherein the flexion is a second flexion comprising a deformation of the upper lobe to complement the cavity to fit the earpiece in the ear.

15. The method of claim 11, wherein the earpiece further comprises an elongate rib extending between the upper and lower ends of the arcuate rib.

16. The method of claim 15, wherein:
the aperture is a first aperture;
the earpiece further comprises a second aperture disposed in at least one of the arcuate rib and/or the elongate rib and configured to receive an extension passing therethrough; and
the method further comprises inserting at least a portion of the extension into an ear canal of the ear.

17. The method of claim 16, wherein:
the earpiece further comprises the extension;
the upper lobe and the extension are biased toward each other forming a pair of clamp jaws; and
the method further comprises clamping the earpiece against the ear by the clamp jaws to secure the earpiece in the ear.

18. The method of claim 15, wherein:
the arcuate rib and the elongate rib form a continuous loop defining an interior void;
the earpiece further comprises a membrane disposed across the interior void; and
the method further comprises attenuating, by the membrane, sound passing from an external environment to the interior void.

19. The method of claim 15, wherein the arcuate rib, the upper lobe, and the elongate rib comprise a single continuous molded workpiece.

20. The method of claim 15, wherein at least a portion of the arcuate rib and/or at least a portion of the elongate rib is substantially hollow.
